(19) United States
(12) Patent Application Publication
(10) Pub. No.: US 2010/0015198 A1
(43) Pub. Date: Jan. 21, 2010

(54) TRANSPLANTS ENCAPSULATED WITH SELF-ELASTIC CARTILAGE AND METHOD OF PREPARING THE SAME

(76) Inventor: Jeong Ik Lee, Seoul (KR)

Correspondence Address: HOLME ROBERTS & OWEN LLP, 1700 LINCOLN STREET, SUITE 4100, DENVER, CO, 80203, US

(21) Appl. No.: 12/306,470

(22) PCT Filed: Jun. 26, 2007
(86) PCT No.: PCT/KR07/03091

§ 371 Date: Jun. 29, 2009

(30) Foreign Application Priority Data
Jun. 26, 2006 (KR) 10-2006-0057252

(51) Int. Cl. A61F 2/02; A61K 35/12

(52) U.S. Cl. 424/423; 424/93.7

(57) ABSTRACT

Disclosed is an implantable microparticle in which a transplant is encapsulated with elastic cartilage derived from the subject receiving the transplant. Also disclosed is a method of preparing an implantable microparticle, comprising (1) isolating elastic cartilage from a subject receiving a transplant; (2) multiplying the elastic cartilage through subculture; (3) mixing the elastic cartilage and the transplant, and subjecting a resultant mixture to shaking culture in order to allow the elastic cartilage to become attached around the transplant; and (4) isolating microparticles in which the transplant is encapsulated with the elastic cartilage.

### TECHNICAL FIELD

The present invention relates to a transplant encapsulated with a recipient's own elastic cartilage and a method of preparing the same. More particularly, the present invention relates to an implantable microparticle in which a transplant is encapsulated with elastic cartilage derived from a subject receiving the transplant, and a method of preparing an implantable microparticle. The method comprises isolating elastic cartilage from a subject receiving a transplant; multiplying the elastic cartilage through subculture; mixing the elastic cartilage and the transplant, and subjecting the resultant mixture to shaking culture in order to allow the elastic cartilage to become attached around the transplant; and isolating microparticles in which the transplant is encapsulated with the elastic cartilage.

### BACKGROUND ART

Islet transplantation is a new therapy for severe insulin-dependent diabetes mellitus (type I diabetes). This method enables the treatment of diabetic complications that cannot be solved only through insulin administration, including renal failure, retinopathy, neuropathy and foot ulcers, without complication. For this reason, islet transplantation is receiving interest as a radical therapy, and an increasing number of clinical trials are reported every year in the world.

However, there are many obstacles that have yet to be overcome, including a shortage of donor organs and regulation of immunorejection of islet transplants after transplantation. At present, immunosuppressive drugs need to be administered for the rest of a patient's life. However, lifelong immune suppression places enormous economic burden on the patient, and these drugs have some serious side effects. In particular, the significant drawback is that immunosuppressive drugs negatively affect transplanted islets in a direct manner.

In order to avoid the need for the lifelong immunosuppressive drugs, immunoisolation has been developed. Immunoisolation is a technique based on encapsulating a transplant to be transplanted using a selectively permeable membrane which allows the permeation of smaller molecules, such as oxygen, CO₂, glucose, amino acids, and hormones, but prevents the penetration of immunocytes and larger immune molecules, such as antibodies and complements. If this concept is ideally achieved, islet transplantation is achievable without immune rejection in a transplanted patient.

The immunoisolation technique used, in the early stages, artificial synthetic materials such as agarose, alginate, gelatin, poly(L-lysine), HEMA-MMA, polyvinyl alcohol, polyglycolic acid and polytetrafluoroethylene polymers, to make a membrane for enveloping organs or tissues. However, these synthetic materials are substantially unlikely to be clinically applicable because they have low in vivo biocompatibility and durability and cause foreign body reactions themselves.

To replace the artificial synthetic materials, cartilage cells have been selected. Cartilage tissue lacks blood vessels, nerves or lymph vessels in normal states, and is thus impermeable to inflammatory cells such as leukocytes. In particular, elastic cartilage rapidly bounces back to its original form when bent, owing to its great flexibility and elasticity. These properties are beneficial in that elastic cartilage can serve as a membrane encapsulating biological cells or tissues. Even as people age, cartilage tissue does not harden because it contains large amounts of collagen, elastin, proteoglycan, etc. as extracellular substrates. Of these, collagen fibers are matured and stabilized by two non-reducible cross-liking substances, pyridinoline and histidino-alanine.

International Patent Application WO 96/40887 discloses a technique for encapsulating islets with cartilage from knee joints. However, since this method is based on loading islets for transplantation onto a biodegradable polyglycolic acid polymer and wrapping the islets with a cartilage membrane, it still employs an artificial material such as the polymer, which can cause a foreign body reaction. In particular, the cartilage membrane is provided in a monolayer form, and thus has very low mechanical integrity. The polymer as well as islets is highly liable to penetrate the thin cartilage membrane, resulting in serious foreign body reactions and immunorejection. The method also has other problems leading to difficulty in substantial clinical use, as follows. Encapsulation using the cultured cartilage membrane is carried out merely by laying islets onto the recovered cartilage membrane and allowing the cartilage membrane to wrap the islets, without an additional process (such as pressing with a heavy article in a process for producing a sheet-type artificial pancreas, or the use of adhesive factors, which are detached along with the cartilage upon cartilage recovery and aid encapsulation). For this reason, islets are not tightly attached to the cartilage membrane. Also, the monolayered cartilage membrane is recovered by artificially detaching a cartilage cell membrane grown to confluency using a cell scraper. This cell detachment damages the weak monolayer and collagen matrices produced by chondrocytes. Further, the use of articular knee cartilage may cause severe side effects on the knee.

Due to the drawbacks in the use of articular knee cartilage, the ear is preferred as a cartilage tissue supply organ. In particular, ear cartilage (elastic cartilage) has already been proven safe and easy to use because deformation of the ear after the excision of the auricular cartilage is rarely observed in clinical plastic surgery field. Therefore, the collection of auricular cartilage as the source of immunoisolation material is acceptable for clinical application in view of cosmetics. However, rodents such as rats have very small auricle and auditory canals, which serve as an elastic cartilage source, and are thus rarely used in animal tests. Since dogs have relatively large amounts of elastic cartilage, so that pure cartilage tissue can be more readily obtained from dogs than rats, animal tests are underway using dogs. However, animal tests are generally conducted by carrying out a basic experiment using rodents and then testing in a larger animal, in this case dogs, based on the first test results. In particular, compared to rats, dogs require a longer testing period, and make it difficult to perform accurate and rapid tests.

On the other hand, the present inventors, prior to this application, developed a macroencapsulated bioartificial pancreas using cell sheet engineering, which comprises culturing chondrocytes extracted from the ear of a dog in a sheet form, and inserting pancreatic islet cells extracted from another dog or a rodent between sheets in order to allow the pancreatic islet cells to be enclosed by chondrocytes and collagen secreted therefrom. However, in the case of rodents, when subcultured through repeat culturing, chondrocytes are mostly contaminated with fibroblasts and are thus not formed in a sheet The fibroblasts in part weaken the association between cells, causing pores during recovery of the chondrocyte sheet. Also, fibroblasts divide more actively than chondrocytes during the proliferation of chondrocytes, resulting in a decrease in the purity of chondrocytes. Thus, since chondrocytes from rodents are difficult to provide in a sheet form, cartilage was collected from dogs. In addition, the bioartificial pancreas is prepared through a complicated and time-consuming process comprising multilayering the sheet. In particular, a monolayered cartilage sheet needs to be handled with skilled techniques because it has technical difficulties such as the formation of pores during handling and the sheet becoming crumpled or rolled at its end. The sheet is multilayered by laying a heavy-weight article onto an upper part of the sheet to press the sheet. This pressing affects islets within the sheet, and islets become flat due to the heavy load. The flat islets exert functional troubles with decreased insulin release. In addition, during the preparation of a bioartificial organ using a chondrocyte sheet, the temperature should be continuously maintained at 37° C. Even when a culture medium is exchanged and a culture is microscopically observed, the temperature should not be below 37° C. Further, the bioartificial organ thus obtained has another problem in that it is large and thus needs a large incision corresponding to its large size when directly implanted in the body.

### DISCLOSURE OF THE INVENTION

In order to provide an immunoisolated transplant, which is effective and substantially clinically applicable, the present inventors prepared microparticles in which a transplant is encapsulated with elastic cartilage by isolating elastic cartilage from a subject receiving the transplant, multiplying the elastic cartilage, mixing the elastic cartilage and the transplant, and subjecting the mixture to shaking culture in order to allow the elastic cartilage to become attached around the transplant. The present inventors found that, since the microparticles are chondrocytes derived from the recipient, they are not recognized as “non-self” but as “self” by the immune system of the recipient, and that the chondrocytes prevent infiltration by cells and immune molecules such as complements, thereby preventing immunorejection, while freely allowing the diffusion of nutrients and gases, and maintaining the original functions of the transplant for a long period of time.

It is therefore an object of the present invention to provide an implantable microparticle in which a transplant is encapsulated with elastic cartilage derived from the subject receiving the transplant.

It is another object of the present invention to provide a method of preparing an implantable microparticle, comprising (1) isolating elastic cartilage from a subject receiving a transplant; (2) multiplying the elastic cartilage through subculture; (3) mixing the elastic cartilage and the transplant, and subjecting the resultant mixture to shaking culture in order to allow the elastic cartilage to become attached around the transplant; and (4) isolating microparticles in which the transplant is encapsulated with the elastic cartilage.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a phase-contrast micrograph of islet cells before being encapsulated with elastic cartilage according to an embodiment of the present invention;

FIG. 2 is a phase-contrast micrograph of islet cells after being encapsulated with elastic cartilage according to an embodiment of the present invention;

FIG. 3 shows the result of a histological examination of microparticles, in which islet cells are encapsulated with elastic cartilage, using H&E staining according to an embodiment of the present invention;

FIG. 4A is a graph showing insulin release patterns of microparticles smaller (small microparticles) and larger (large microparticles) than 200 μm, in which islet cells are encapsulated with elastic cartilage, over time according to an embodiment of the present invention;

FIG. 4B is a graph showing insulin release patterns of microparticles, in which islet cells are encapsulated with elastic cartilage, for a long period of time according to an embodiment of the present invention;

FIG. 5 shows the progress of encapsulation of islet cells with elastic cartilage according to another embodiment of the present invention;

FIG. 6 shows microparticles in which islet cells are encapsulated with elastic cartilage according to another embodiment of the present invention;

FIG. 7A shows the result of H&E staining of microparticles, in which islet cells are encapsulated with elastic cartilage, according to another embodiment of the present invention;

FIG. 7B shows the result of immunohistochemical staining of microparticles, in which islet cells are encapsulated with elastic cartilage, for insulin according to another embodiment of the present invention;

FIG. 7C shows the result of dithizone staining of microparticles, in which islet cells are encapsulated with elastic cartilage, according to another embodiment of the present invention;

FIG. 8 is a graph showing insulin release patterns of microparticles, in which islet cells are encapsulated with elastic cartilage, over time according to another embodiment of the present invention; and

FIGS. 9A and 9B show the results of evaluation of the immunoisolation efficacy of microparticles, in which islet cells are encapsulated with elastic cartilage, according to another embodiment of the present invention.

### BEST MODE FOR CARRYING OUT THE INVENTION

In one aspect, the present invention relates to an implantable microparticle in which a transplant is encapsulated with elastic cartilage derived from a subject receiving the transplant.

The term “transplant”, as used herein, refers to a cell derived from an organ or tissue to be implanted, but may be also a micro-sized segment that is obtained by finely sectioning an organ or tissue to be implanted. Since the transplant is provided as a cell unit in the present invention, it may be a cell genetically modified so as to carry a new specific function (e.g., hormone secretion) or to be improved with respect to specific functions.

The type of the transplant is not specifically limited. In one aspect, it is preferable that the transplant be a cell type having an endocrine function because this cell type is expected to have excellent therapeutic effects on a specific disease. For example, when derived from the pancreas or the thyroid gland, the transplant may be used for treating diabetes mellitus or hypothyroidism, respectively. As other examples, when the transplant is a cell secreting erythropoietin, a growth hormone or a blood clotting factor, it may be used for treating anemia, dwarfism or hemophilia, respectively.

In the present invention, the transplant is preferably pancreatic islet cells, which secrete hormones such as insulin and glucagons to regulate blood glucose levels. This is due to the complementary nature of chondrocytes and islet cells. That is, the chondrocytes protect the pancreatic islets by providing extracellular matrices and avoiding disaggregation which diminishes its function, and insulin secreted by islet cells acts as a growth factor for chondrocytes.

“Elastic cartilage” encapsulating the transplant refers to cartilage isolated from the ear of the subject receiving the transplant. Chondrocytes are present in several parts of the body, but chondrocytes from the ear are suitable because they are easy to isolate and proliferate relative to those in other parts of the body, do not cause side effects such as functional defects in the body, and leave no scars.

The transplant is primarily encapsulated with elastic cartilage, which is attached to the transplant at areas surrounding the transplant, but the transplant may be considered to be substantially encapsulated with a membrane consisting of collagen because the attached elastic cartilage secretes collagen. The membrane has a plurality of fine pores. Since the pores have a sufficiently small size, ranging from 50 nm to 200 nm, cells and immune molecules, such as complements, cannot penetrate through the pores, but the pores are permeable to gases, such as O₂ and CO₂, nutrients, such as glucose and amino acids, and substances secreted by the transplant. Thus, the transplant exerts its innate functions without immune rejection by the host immune system.

A microparticle, in which the transplant is encapsulated with elastic cartilage, has a size from 150 μm to 800 μm and is suitable for therapeutic purposes. It is more preferable that the microparticle has a small size of about 200 μm. This is because the encapsulation of the transplant with relatively thin elastic cartilage further facilitates the diffusion of gases and nutrients, extends transplant survival, and promotes transplant proliferation, thereby being more beneficial for the transplant to exert its innate functions. However, when the microparticle has an extremely small size, less than 150 μm, attention must be paid to ensure that the microparticle is not a small lump of elastic cartilage that does not contain the transplant in the inside.

In another aspect, the present invention relates to a method of preparing an implantable microparticle, comprising (1) isolating elastic cartilage from a subject receiving a transplant; (2) multiplying the elastic cartilage through subculture; (3) mixing the elastic cartilage and the transplant, and subjecting the resultant mixture to shaking culture in order to allow for the elastic cartilage to become attached around the transplant; and (4) isolating microparticles in which the transplant is encapsulated with the elastic cartilage.

At step (1) of the present method, elastic cartilage is isolated from the subject receiving a transplant. First, elastic cartilage of the auditory canal and auricle of the ear is collected from the recipient subject After skin tissue, subcutaneous tissue, muscular tissue, perichondrium and other connective tissues are eliminated, elastic cartilage is finely chopped using physical means, such as a homogenizer, a mortar, a blender, a surgical scalpel, syringes, forceps or an ultrasonication device. At this time, elastic cartilage is preferably placed onto a watch glass and chopped using curved scissors. Curved scissors are provided in a bent form, which facilitates close contact with the concave watch glass. The cutting using curved scissors continues while the watch glass is rotated. This circular motion gathers elastic cartilage in the center, and the cutting motion of the curved scissors, effective against elastic cartilage, is continuously applied, resulting in finely chopped elastic cartilage in a much smaller size within a short time. When elastic cartilage is chopped as finely as possible, it has a larger surface area contacting a digestion enzyme at a post-step, ensuring more effective digestion and shortening digestion time.

After being finely chopped, elastic cartilage is digested with at least one protease selected from among neutral proteases, trypsin, serine proteases, elastases and collagenases. The digestion is carried out with agitation at the same temperature as the body temperature of the recipient, overnight or for a long period of three to five days. The digestion temperature and time may vary depending on the type of proteinase, the species of the recipient, and the like, but it is preferable that, when little elastic cartilage is taken from the recipient, it is treated with a digestion enzyme for a longer period of time in order to obtain pure chondrocytes. The reasons are as follows. During the digestion using a protease, chondrocytes and other cells (muscle cells, fibroblasts, etc.) are disaggregated into single cells by the action of the protease. Since the digestion is carried out with agitation in the present invention, the separated single cells are maintained in a suspended state. The suspended state is unfavorable for adhesive cells, which are able to proliferate only when attached onto the bottom of culture dishes. Over time, rapidly metabolizing cells die, and eventually, only chondrocytes having low metabolic rates survive. Thus, it is possible to isolate only chondrocytes in high purity.

At step (2) of the present method, the isolated elastic cartilage is multiplied through subculture. Any known medium for chondrocyte proliferation may be used. The medium is essentially supplemented with ascorbic acid required for collagen synthesis, and is optionally supplemented with a proliferation factor, such as FGF (fibroblast growth factor), HGF (hepatocyte growth factor) and IGF (insulin-like growth factor). When elastic chondrocytes are subcultured for a period of about four weeks, it is preferable to use a medium capable of multiplying from 15,000 to 130,000 times, the medium consisting of CBM (CAMBREX. Co.), CGM SingleQuots (CAMBREX Co.) and ascorbic acid. When the culture reaches about 90% confluency, the multiplied chondrocytes are detached with trypsin-EDTA, and subcultured in a fresh medium. The number of subcultures is preferably limited to three times or less. As the subculturing is continued, particularly after the 4th passage, chondrocytes are dedifferentiated, losing their innate nature into fibroblast-like cells, in which the synthesis of collagen as a secretory extracellular substrate is switched from type II to type I. When chondrocytes are cultured for a period of about four weeks (required to obtain cells before the 4th passage of subculture) under the aforementioned conditions, they may be multiplied a minimum of 15,000 times.

At step (3) of the present invention, the elastic cartilage and transplant are mixed and subjected to shaking culture in order to allow for the elastic cartilage to become attached around the transplant. At this time, the elastic cartilage should be added in an excessive amount to sufficiently encapsulate the transplant. Due to the adhesive nature of the cells, the oscillatory motion artificially increases contact frequency between cells. This results in a large amount of elastic cartilage being rapidly attached onto the transplant surrounding the transplant, thereby growing microparticles. In particular, when chondrocytes exist at high density, they return to a state similar to the original state of cartilage tissue while maintaining their size. That is, chondrocytes come to abundantly produce and secrete type II collagen, which is an innate extracellular substrate, and thus have the same properties as in original in vivo chondrocytes, which have a low cell density and contain plenty of extracellular substrates.

At step (3), a medium for insulin release analysis may be used. The medium is preferably prepared by supplementing RPMI glucose(−) medium and/or Ham's F-12 medium with 0.8 mg/ml to 1.2 mg/ml of glucose, 10% heat-inactivated FBS, HEPES, ascorbic acid, an antibacterial agent and an antifungal agent. The shaking culture is preferably carried out at the same temperature as the body temperature of the recipient for a period of three to ten days at 60 to 80 rpm using a shaker, the platform of which moves in a three-dimensional “8”-shaped fashion or a planar circular or linear fashion. The agitation speed may vary depending on the diameter of a culture container. A non-adhesive culture dish (that is, a dish for suspension culture), which is designed to prevent the adhesion of cells to the dish surface, or a spinner flask is employed. For example, a suitable culture dish is a HydroCell™ culture dish (CellSeed. Co.), to which cells never attach, due to the dish's hydrophilicity at about 37° C. This step yields microparticles between about 150 μm to 800 μm.

At step (4) of the present method, microparticles in which the transplant is encapsulated with the elastic cartilage are isolated. When the shaking culture is continued for a period of three to ten days at step (3), microparticles of various sizes are present in the culture dish. In order to simply isolate microparticles or to isolate microparticles according to the size of the microparticles, in one aspect, the microparticles may be observed under a phase-contrast microscope and may be suctioned using a sterile micropipette, and transferred into new culture dishes (for suspension culture). At this time, culture dishes are swirled to gather the microparticles to the center of the dishes by centrifugal force. The microparticles are then suctioned along with the culture medium using a micropipette tip, thereby obtaining only microparticles having a size smaller than the diameter of the tip. The isolated microparticles may be stored in a medium for insulin release analysis.

A better understanding of the present invention may be obtained through the following examples, which are set forth to illustrate, but are not to be construed as the limit of the present invention.

### Example 1

### Preparation of Microparticles in which Rat Islet Cells are Encapsulated with Dog or Rat Chondrocytes and Evaluation of Insulin Release from the Microparticles

1-A. Isolation and Culture of Chondrocytes

Elastic cartilage from the auditory canal and auricle of the ear was collected from beagles (12 to 24 months old) and Brown Norway rats (weighing 350±50 g). After skin tissue, subcutaneous tissue, muscular tissue, perichondrium and other connective tissues were eliminated, the elastic cartilage was placed onto a watch glass and finely chopped using curved scissors. A digestion solution was prepared by dissolving 0.3% (for dogs) or 0.15% (for rats) collagenase class II (Worthington, Biochemical Co.) and 0.25% trypsin (Invitrogen Co.) in Ham's F-12 medium (Gibco. Co.) supplemented with 10% heat-inactivated FBS (fetal bovine serum), HEPES, 4% antibacterial/antifungal mixture (10,000 units/ml of penicillin G, 10,000 μg/ml of streptomycin sulfate and 25 μg/ml of amphotericin B; Invitrogen Co.) and 50 μg/ml of ascorbic acid. The elastic cartilage obtained from dogs and rats was treated with the digestion solution and allowed to digest overnight in a shaking water bath at 37° C. in order to be dissociated into single cells. After the digestion was completed, the cell suspension was sequentially filtered through 70-μμm and 40-μm nylon cell-strainers (BD Falcon™, BD Biosciences), and was then washed twice with PBS (Invitrogen Co.) containing 4% antibacterial/antifungal mixture as described above.

The thus obtained single cells were seeded in a proliferation medium to multiply the number of chondrocytes. In the case where they were derived from dogs, 0.5×10⁴ cells were seeded first, and 0.25×10⁴ cells were then seeded in subsequent subculture. In the case where they were derived from rats, 1.0×10⁴ cells were seeded first and 0.5×10⁴ cells were then seeded in subsequent subculture. The proliferation medium consisted of CBM (CAMBREX. Co.), CGM SingleQuots (CAMBREX. Co.) and 50 μg/ml of ascorbic acid. When the culture reached about 90% confluency, the proliferated chondrocytes were detached from culture dishes with trypsin-EDTA.

1-B. Isolation of Islet Cells

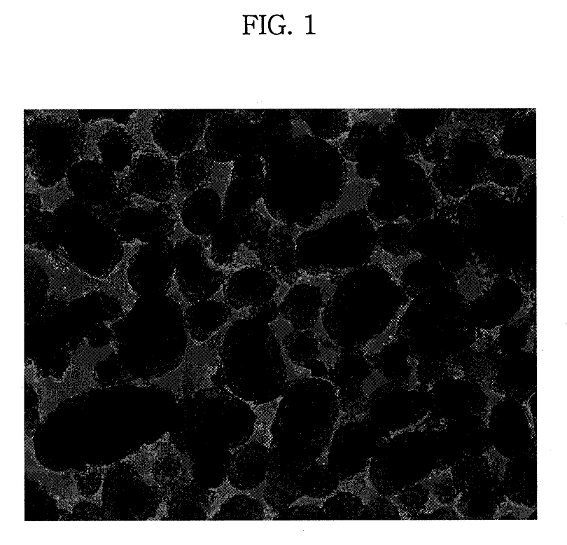

The pancreas was excised from Brown Norway rats or Lewis rats (weighing 350±50 g). 2 mg/ml of collagenase P (Roche. Co.) was added to Hanks' Balanced Salt Solution (HBSS; Gibco. Co.) supplemented with 10% heat-inactivated FBS (JRH Biosciences) and 10 mmol/L of HEPES, thus giving a collagenase P solution. Then, the rat pancreas was subjected to collagenase digestion using the collagenase P solution, and was then purified on a Histopaque density gradient (Sigma Co.). The islet cells thus isolated were observed under a phase-contrast microscope. The results are given in FIG. 1.

1-C. Encapsulation of Islets with Chondrocytes

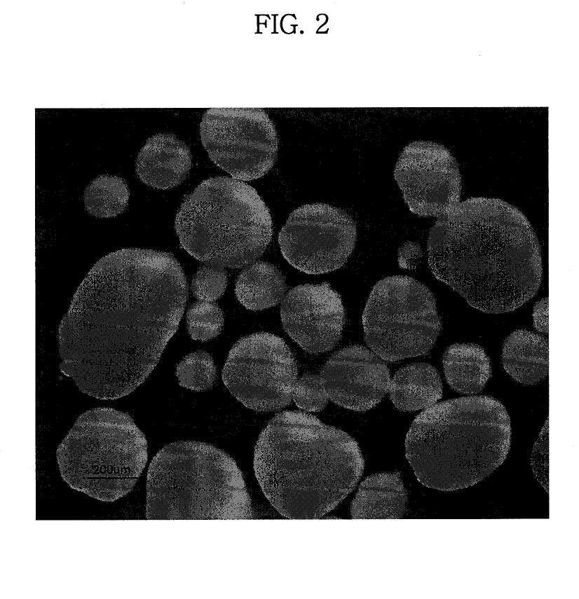

Dog and rat chondrocytes obtained in Example 1-A from the third passage and the first passage of subculture, respectively, were individually resuspended in 5 ml of a medium for insulin release analysis, and adjusted to a density of 1,500×10⁴ cells/5 ml. The resuspended chondrocytes were mixed with the islet cells obtained in Example 1-B, and then cultured in a culture container, 60-mm HydroCell™ (CellSeed. Co.) using a shaker for shaking culture (Model: NA-201, Nissin Co.) at 37° C. and 70 rpm for 7 days. The medium for insulin release analysis contained 50% Ham's F-12 medium Invitrogen Co.) and 50% RPMI-1640 (Invitrogen Co.) containing 25 mmol/L of HEPES, and was supplemented with 1% antibacterial/antifungal mixture as described above, 10% heat-inactivated FBS and 1.0 mg/ml of D-(+)-glucose (Sigma Co.). Microparticles were observed with the naked eye after 12 hrs of culture. They were observed under a phase-contrast microscope, and the results are given in FIG. 2.

The microparticles were present in various sizes. In order to isolate microparticles smaller than 200 μm, the culture dish was swirled to gather the microparticles to the center of the dish by centrifugal force. The microparticles were then suctioned along with the culture medium using a 200-μm micropipette tip, thereby isolating microparticles smaller than 200 μm. This step was repeated, and then the isolated microparticles were transferred into two new suspension culture dishes and fed with a medium for insulin release analysis.

1-D. Histological Evaluation

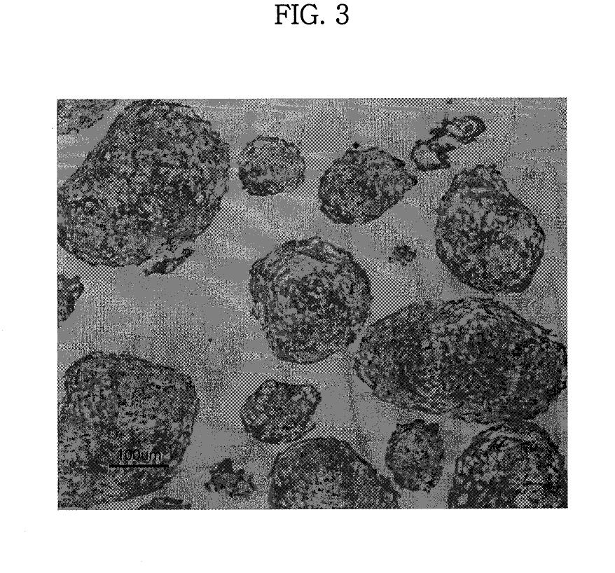

The microparticles obtained by shaking culture for 7 days in Example 1-C were fixed in 4% paraformaldehyde, washed with PBS (Invitrogen Co.), and immersed in PBS containing 15% and 20% sucrose. Subsequently, the microparticles were immediately frozen, embedded in OCT compound (Sakura Finetechnical Co. Ltd.), and sectioned into 5-μm thickness. The sections were stained with hematoxylin and eosin. The results are given in FIG. 3.

1-E. Insulin Measurement

The microparticles were assessed for insulin release levels for another period of about two weeks, excluding the 7-day period for their preparation. This assay was performed using microparticles less than 200 μm (small microparticles) and microparticles greater than 200 μm (large microparticles). The microparticles were placed on a common culture dish and incubated in the medium for insulin release analysis, used in Example 1-C, at 37° C. under 5% CO₂. The culture medium was collected every 24 hrs, and insulin levels were measured using a microparticle enzyme immunoassay, IMXTM Insulin•Dynabac® (Abbot Laboratories Tokyo JAPAN). The insulin content of the medium samples was measured for 13 days.

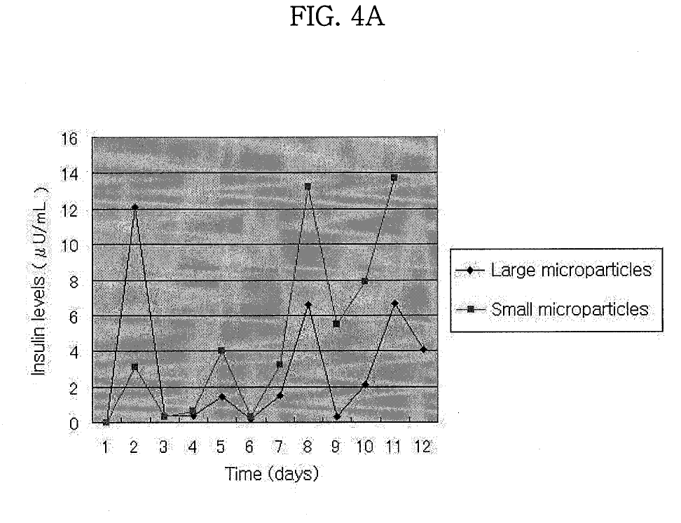

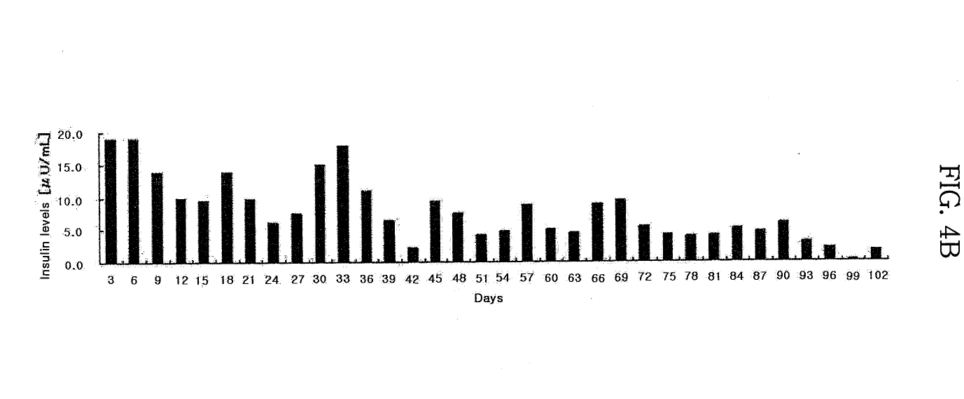

As shown in FIG. 4A, the insulin release from the microparticles was observed for the test period of 13 days, and insulin levels increased after 8 days. After 3 days, microparticles less than 200 μm (small microparticles) displayed higher insulin secretion than microparticles greater than 200 μm (large microparticles).

Separately, 300×10⁴/ml chondrocytes from dogs, obtained in Example 1-A, were mixed with the islet cells obtained in Example 1-B. Then, microparticles were prepared according to the same method as Example 1-C, and assessed for insulin release levels for a long period of time. The insulin levels were measured for a long period of about three months (102 days), excluding a 6-day period for their preparation. The microparticles obtained according to Example 1-C were seeded in three Transwell® inserts (Corning Costar, Corning), which were then placed into 60-mm suspension culture dishes. After the medium for insulin release analysis, used in Example 1-C, was added to the culture dishes, the culture dishes were incubated at 37° C. under 5% CO₂. The culture medium was collected every 72 hrs, and insulin levels were measured using an immunoradiometric assay kit (INSULIN•RIABEAD®; SRL, Inc.). The insulin content of the medium samples was measured every 72 hrs from Days 3 to 102. The results are given in FIG. 4B.

As shown in FIG. 4B, the insulin release from the microparticles was observed for the test period of 102 days. The initial insulin level measured at Day 3 decreased over time, but this decrease was considered small and thus not significant. Compared to the culture of islets alone in which islets typically survive with insulin secretion for only about two weeks under general culture conditions, the present microparticles seemed to greatly enhance the insulin secretion capacity and culture characteristics of islets.

### Example 2

### Preparation of Microparticles in which Rat Islet Cells are Encapsulated with Rat Chondrocytes and Evaluation of the Microparticles for Islet Immunoisolation

2-A. Preparation of Rats

Islets were isolated from adult male Lewis rats (weighing 300±50 g), and auricular cartilage tissue was obtained from male Brown Norway rats (250±50 g; SLC Japan Co.) All rats were intramuscularly administered with a mixture of medetomidine (100 μg/kg) and midazolam (0.5 mg/kg). For anesthesia, ketamine HCl (40 mg/kg) was intramuscularly injected into rats. Animal care and animal experimentation were conducted according to the guidelines of the Graduate School of Agricultural and Life Sciences, the University of Tokyo.

2-B. Isolation and Culture of Islet Cells

A collagenase P solution was prepared by adding 2 mg/ml of collagenase P (Roche. Co.) to cold Hanks' Balanced Salt Solution (HBSS; Gibco. Co.) supplemented with 5% heat-inactivated FBS (JRH Biosciences) and 10 mmol/L of HEPES. Then, the common pancreatic duct was cannulated and perfused with the collagenase solution to expand the pancreas. The expanded pancreas was digested in a shaking water bath at 37° C. for 16 min. The digestion product was filtered through a 600-μm steel mesh, and was then purified on a Histopaque density gradient (Sigma Co.). Thereafter, islet cells were cultured overnight in a medium for insulin release analysis, which contained 50% Ham's F-12 medium Invitrogen Co.) and 50% RPMI-1640 (Invitrogen Co.) containing 25 mmol/L of HEPES, and supplemented with 1% antibacterial/antifungal mixture (10,000 units/ml of penicillin G, 10,000 μg/ml of streptomycin sulfate and 25 μg/m of amphotericin B; Invitrogen Co.), 50 μg/ml of ascorbic acid, 10% heat-inactivated FBS and 1.0 mg/ml of D-(+)-glucose (Sigma Co.).

2-C. Isolation and Culture of Auricular Chondrocytes

Auricular cartilage was collected from Brown Norway rats. After skin tissue, subcutaneous tissue, muscular tissue, perichondrium and other connective tissues were removed, the auricular cartilage was placed onto a watch glass and finely chopped using curved scissors. A digestion solution was prepared by dissolving 0.15% collagenase class II (Worthington, Biochemical Co.) and 0.25% trypsin (Invitrogen Co.) in Ham's F-12 medium (Gibco. Co.) supplemented with 10% heat-inactivated FBS, HEPES, 4% antibacterial/antifungal mixture as described above and 50 μg/ml of ascorbic acid. Then, the auricular cartilage was treated with the digestion solution and allowed to digest in a shaking water bath at 37° C. After the digestion was completed, the cell suspension was sequentially filtered through 70-μm and 40-μm nylon cell-strainers (BD Falcon™, BD Biosciences), and was then washed twice with PBS (Invitrogen Co.) containing 4% antibacterial/antifungal mixture as described above.

The chondrocytes thus obtained were cultured in a medium consisting of CGM SingleQuots (CAMBREX. Co.), CBM™ (CAMBREX. Co.) and 50 μg/ml of ascorbic acid at 37° C. under humidified 5% CO₂. The chondrocytes were inoculated at a density of 1.0×10⁴ cells/cm² first, and at a density of 0.5×10⁴ cells/cm² in subsequent subculture. The medium was exchanged twice every a week. The number of subcultures was limited to one time. When the culture reached about 90% confluency, the proliferated chondrocytes were detached from culture dishes with trypsin-EDTA, and used in the islet encapsulation described below.

2-D. Encapsulation of Islet Cells with Chondrocytes

The islet cells obtained in Example 2-B were plated onto a culture dish, 60-nm HydroCell™ (CellSeed. Co.). The chondrocytes were then resuspended at a density of 1,500×10⁴ cells/5 ml of medium for insulin release analysis, and were added to the culture dish, followed by shaking culture. The shaking culture was carried out using a shaker for shaking culture (NA-201; Nissin Co.), which operated on a horizontal plane at 70 rpm, for six days, thereby yielding microparticles in which the islet cells were encapsulated with the chondrocytes. As a control, without the addition of chondrocytes, only islet cells were cultured with agitation (hereinafter, the islet cells were referred to as “nude islet cells”).

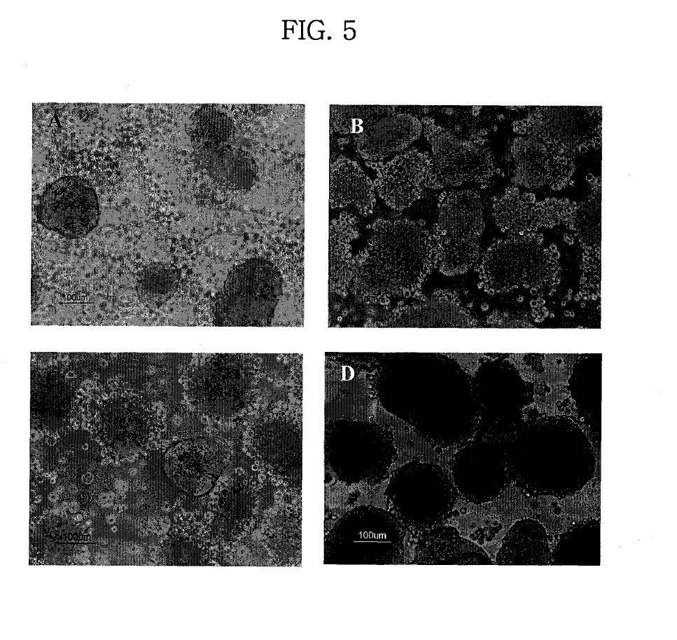

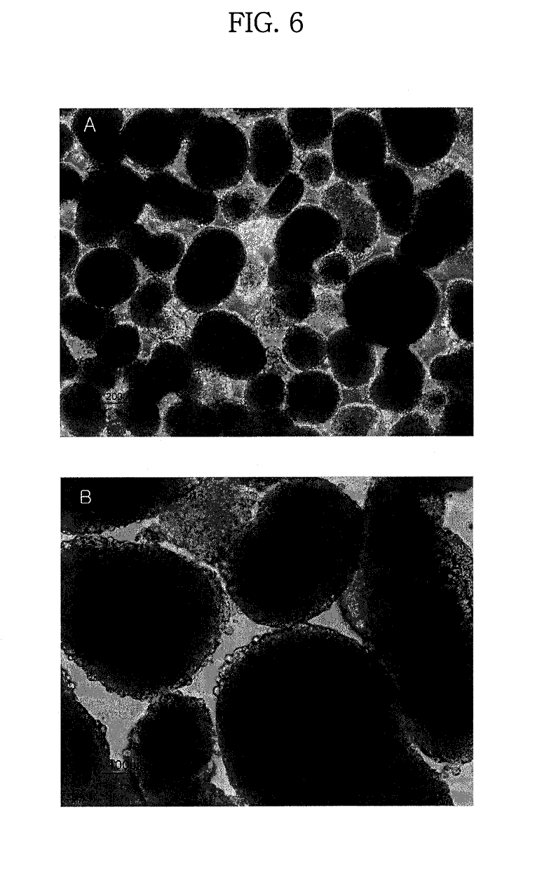

The microparticles obtained at given time points were observed under a phase-contrast microscope, and the results are given in FIG. 5. In FIG. 5, panels A, B, C and D show microparticles collected at the culture starting point, after 30 hrs, after 51 hrs and after 99 hrs, respectively. Chondrocytes were observed to attach onto islet cells, surrounding the islet cells, and the appearance of microparticles gradually became smooth over time. Also, after 125 hrs of culture, microparticles were collected and observed under a phase-contrast microscope, and the results are given in FIG. 6 (A: 40 times magnified, B: 100 times magnified). Small microparticles were 250±100 μm in diameter, and large microparticles were 600±200 μm in diameter.

2-E. Histological Evaluation

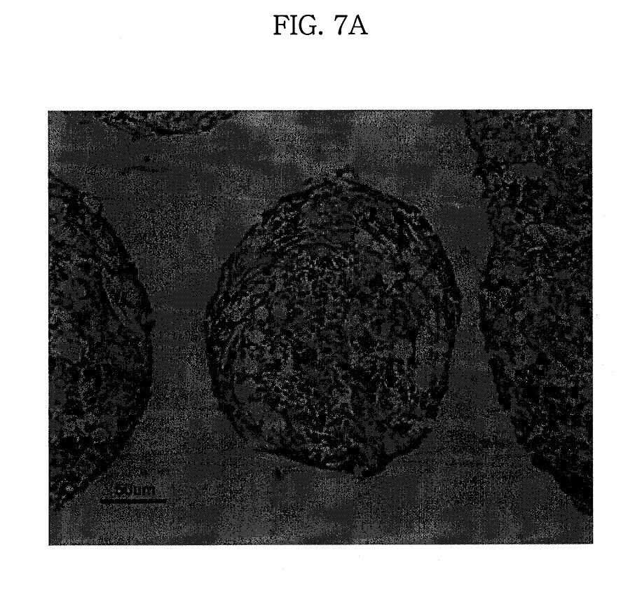

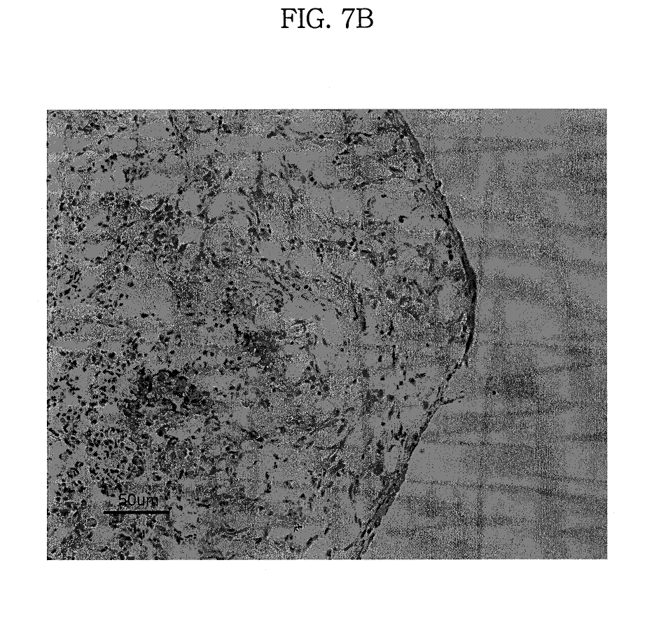

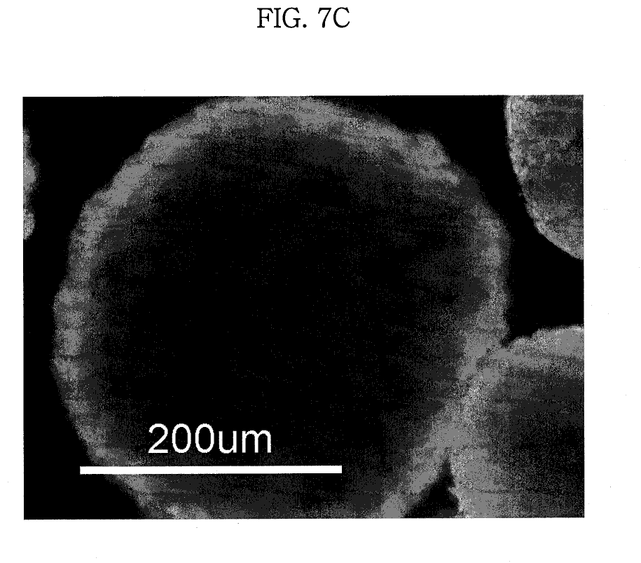

The microparticles obtained by shaking culture for 6 days in Example 2-D were fixed in 4% paraformaldehyde, washed with PBS (Invitrogen Co.), and immersed in PBS containing 15% and 20% sucrose. Subsequently, the microparticles were immediately frozen, embedded in OCT compound (Sakura Finetechnical Co. Ltd.), and sectioned to a 5-μm thickness. The sections were stained with hematoxylin and eosin. The results are given in FIG. 7A.

In addition, the microparticles were immunohistochemically stained for insulin using an avidinbiotin-peroxidase complex technique (LSAB 2 kit/HRP, DAKO Japan Co., Ltd.), which employs 3-amino-9-ethylcarbazole (AEC) substrate-chromogen solution (DAKO Japan Co., Ltd.), according to the company's protocol. The results are given in FIG. 7B. The densely stained areas indicated the presence of insulin. These results revealed that islet cells were present within the microparticles and had intact insulin secretion ability.

Further, dithizone staining was performed to identify viable islet cells incorporated in the microparticles, and the results are given in FIG. 7C. Dithizone binds selectively to pancreatic β-cells. As shown in FIG. 7C, more densely stained areas were found in a microparticle, confirming that islet cells were incorporated within the microparticles.

2-F. In Vitro Evaluation of Immunoisolation Capacity of the Microparticles

The microparticles were seeded in twelve Transwell® inserts (Corning Costar, Corning), and were divided into three test groups including control group. As a control, nude islet cells were seeded in six Transwell® inserts.

Separately, sera were collected from one healthy Beagle (male, 15.5 kg, 6-year-old), and immediately cryopreserved at −80° C. until use. Two kinds of media were used for the evaluation of complement-dependent cytotoxicity. The media had the same composition as in the medium for insulin release analysis, except that 10% heat-inactivated FBS was replaced by 10% dog serum (referred herein to as “MCM-Dog medium”; containing xenogenic complements) or 10% Lewis serum (referred herein to as “MCM-Lewis medium”; containing allogenic complements). Microparticles classified into a xenogenic group (n=6) were cultured in MCM-Dog medium, and microparticles classified into an allogenic group (n=6) in MCM-Lewis medium. A nude group (n=6; nude islet cells) as a control was cultured in MCM-Lewis medium. After 72 hrs, the insulin content of the culture medium was first measured to indirectly estimate the activity of complements. The culture medium was collected every 72 hrs and centrifuged. The supernatants were assessed for insulin levels using an immunoradiometric assay kit (INSULIN•RIABEAD® II; SRL, Inc.).

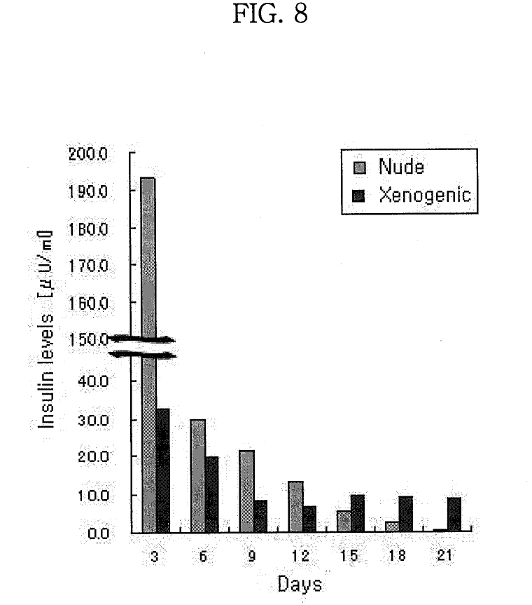

The measured insulin levels are given in FIG. 8. In the case of nude islet cells as a control, released insulin levels dropped from 193.3 μU/ml to 21.5 μU/ml at Day 9 and to 2.4 μU/ml at Day 18. In case of the xenogenic group, initial insulin levels also decreased, but insulin levels slightly increased after Day 12 and remained constant during the rest of the test period of 40 days.

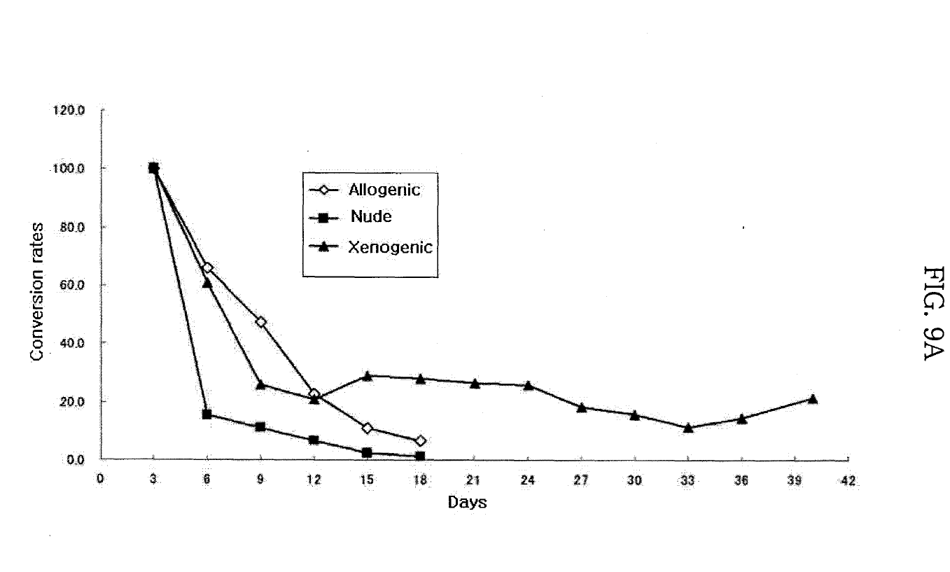

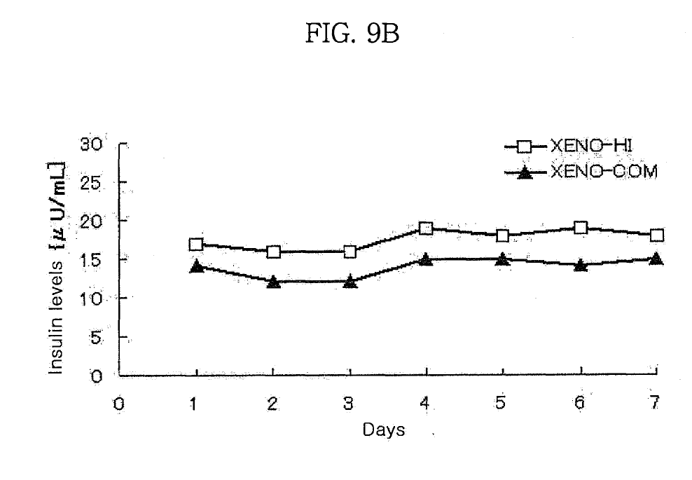

In vitro complement-dependent cytotoxicity was expressed as a percentage of insulin secretion, and the conversion rate was calculated according to the following equation: (the amount of insulin released from microparticles or nude islet cells at a given time point/the amount of insulin released from microparticles or nude islet cells at the initial time point)×100. The results are given in FIG. 9A. In the nude group, the initial insulin release dropped to 11.1% at Day 9 and to 1.3% at Day 18. The xenogenic group also exhibited a decrease in insulin levels, but insulin levels were maintained constant at 20% of the initial insulin level for a test period of 40 days. The allogenic group displayed a slow decrease in insulin release. These results indicate that xenogenic complements did not have any cytotoxic activity toward microparticles.

In order to strictly evaluate xenogenic complement-dependent cytotoxicity, two media different from those used above were used. The media had the same composition as in the medium for insulin release analysis, except that 10% heat-inactivated FBS was replaced by 50% dog serum (referred herein to as “XENO-COM medium”; containing xenogenic complements) or 50% heat-inactivated dog serum (referred herein to as “XENO-HI medium”; since xenogenic complements lose its activity, xenogenic immunorejection is not caused. Microparticles were cultured in each medium. After 24 hrs, the insulin content of the culture medium was measured in order to indirectly estimate complement activity. The culture medium was collected every 24 hrs and centrifuged. The supernatants were assessed for insulin levels using an immunoradiometric assay kit (INSULIN•RIABEAD® II; SRL, Inc.).

FIG. 9B shows the result of the strict evaluation of xenogenic complement-dependent cytotoxicity. Insulin release was maintained at constant levels regardless of heat inactivation of the added xenogenic serum. These results further confirmed the finding that xenogenic complements did not have any cytotoxic activity toward microparticles.

Taken together, these results demonstrate that the microparticles according to the present invention are not destroyed by host immunorejection even upon xenotransplantation.

### INDUSTRIAL APPLICABILITY

As described hereinbefore, since the microparticles according to the present invention are chondrocytes derived from a subject receiving a transplant, they are recognized not as “non-self” but as “self” by the immune system of the recipient subject. Also, since the transplant is encapsulated with chondrocytes, the microparticles prevent infiltration by cells and immune molecules such as complements, thereby preventing immunorejection, while permitting the free diffusion of nutrients and gases, and maintaining the innate functions of the transplant for a long period of time. Further, because immunorejection is prevented, the microparticles do not require lifelong immunosuppressive drugs when transplanted, and allow the use of xenogenic organs, thereby overcoming the lack of supply of donor organs available for transplantation.

1. An implantable microparticle in which a transplant is encapsulated with elastic cartilage derived from a subject receiving the transplant.

2. The implantable microparticle as set forth in claim 1, wherein the transplant is a cell.

3. The implantable microparticle as set forth in claim 1, wherein the transplant is derived from an islet of pancreas.

4. The implantable microparticle as set forth in claim 1, which is from 150 μm to 800 μm in size.

5. A method of preparing an implantable microparticle, comprising:
(1) isolating elastic cartilage from a subject receiving a transplant;
(2) multiplying the elastic cartilage through subculture;
(3) mixing the elastic cartilage and the transplant, and subjecting a resultant mixture to shaking culture in order to allow the elastic cartilage to become attached around the transplant; and
(4) isolating microparticles in which the transplant is encapsulated with the elastic cartilage.

6. The method of preparing the implantable microparticle as set forth in claim 5, wherein, at step (1), the elastic cartilage is chopped and is digested with a protease.

7. The method of preparing the implantable microparticle as set forth in claim 6, wherein the elastic cartilage is placed onto a watch glass and is chopped using curved scissors.

8. The method of preparing the implantable microparticle set forth in claim 6, wherein the elastic cartilage is digested with the protease in conjunction with agitation overnight or for a period of five days.

9. The method of preparing the implantable microparticle as set forth in claim 5, wherein, at step (2), the number of subcultures is limited to three times or less.

10. The method of preparing the implantable microparticle as set forth in claim 5, wherein, at step (3), the shaking culture is carried out using a medium for insulin release analysis.

11. The method of preparing the implantable microparticle as set forth in claim 10, wherein the medium for insulin release analysis contains glucose in an amount of 0.8 mg/ml to 1.2 mg/ml.

12. The method of preparing the implantable microparticle as set forth in claim 5, wherein the shaking culture of step (3) is carried out for a period ranging from three days to ten days.
